(19) United States
(12) Patent Application Publication
(10) Pub. No.: US 2007/0249823 A1
(43) Pub. Date: Oct. 25, 2007

(54) PROCESS FOR PREPARING GEMCITABINE AND ASSOCIATED INTERMEDIATES

(75) Inventors: Vladimir Naddaka, Lod (IL); Eyal Klopfer, Tel-Aviv (IL); Shady Saeed, Haifa (IL); Dionne Montvilisky, Givatayim (IL); Oded Arad, Rehovot (IL); Joseph Kaspi, Givatayim (IL)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Chemagis Ltd., Bnei Brak (IL)

(21) Appl. No.: 11/697,379

(22) Filed: Apr. 6, 2007

Related U.S. Application Data
(60) Provisional application No. 60/793,461, filed on Apr. 20, 2006.

(51) Int. Cl. C07H 19/00

(52) U.S. Cl. 536/28.5; 536/55.3

(57) ABSTRACT

The present invention provides novel intermediates, which preferably include 3-substituted, alkyl 2,2-difluoro-3-hydroxy-3-(2,2-dialkyldioxolan-4-yl)-propionate derivatives, and 3,5-disubstituted-2-deoxy-2,2-difluoro-1-oxo-D-ribose derivatives. The present invention also provides processes for producing such intermediates and processes for producing gemcitabine therewith.

### BACKGROUND OF THE INVENTION

Gemcitabine HCl, marketed by Eli Lilly under the trademark Gemzar®, is a nucleoside analogue having antitumor activity and it belongs to a general group of chemotherapy drugs known as antimetabolites. Gemcitabine exhibits cell phase specificity, primarily killing cells undergoing DNA synthesis and blocking the progression of cells through the G1/S-phase boundary.

Gemcitabine is a synthetic glucoside analog of cytosine, which is chemically described as 4-amino-1-(2-deoxy-2,2-difluoro-β-D-ribofuranosyl)-pyrimidin-2(1H)-one or 2′-deoxy-2′, 2′-difluorocytidine (β isomer). Gemcitabine HCl has the following structure:

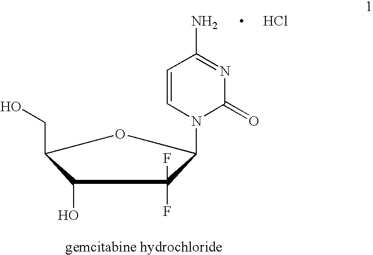

Gemzar® is supplied in vials as the hydrochloride salt in sterile form, only for intravenous use, containing either 200 mg or 1 g of gemcitabine HCl (calculated as free base) formulated with mannitol (200 mg or 1 g, respectively) and sodium acetate (12.5 mg or 62.5 mg, respectively) as a sterile lyophilized powder. Hydrochloric acid and/or sodium hydroxide may have been added for pH adjustment.

U.S. Pat. No. 4,808,614 (“the '614 patent”) describes a process for synthetically producing gemcitabine, which process is generally illustrated in Scheme 1.

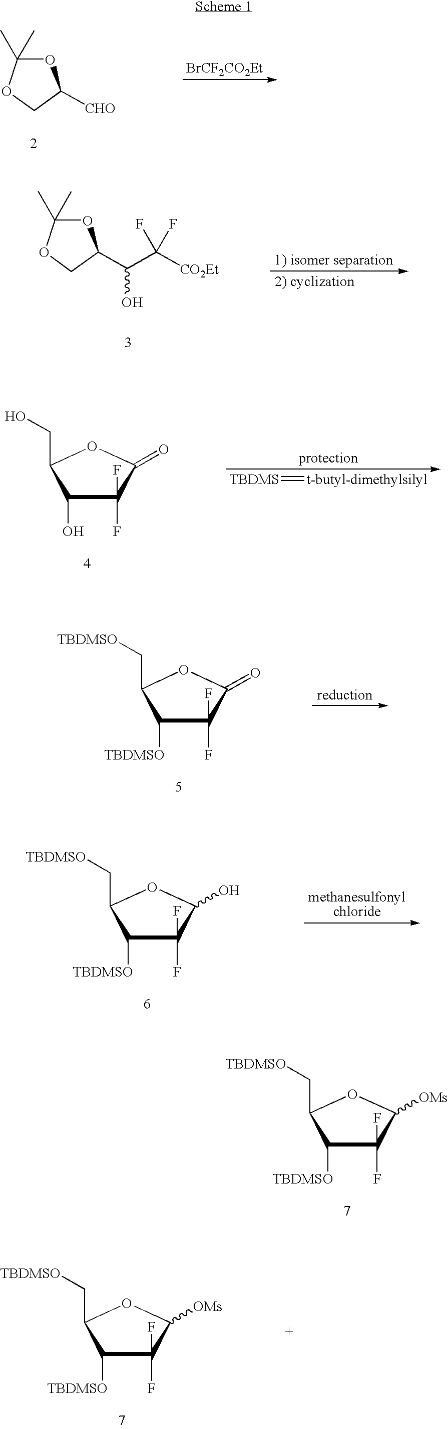

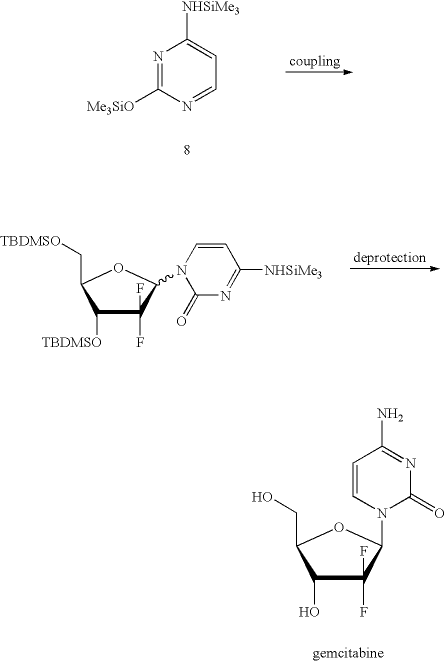

D-glyceraldehyde ketal 2 is reacted with bromodifluoroacetic acid ethyl ester (BrCF₂COOEt) in the presence of activated zinc, to obtain ethyl 2,2-difluoro-3-hydroxy-3-(2,2-dimethyldioxolan-4-yl)-propionate 3 as a mixture of 3-R and 3-S isomers. The 3-R to 3-S isomer ratio is about 3:1. The 3-R isomer has the stereochemistry required for producing desired erythro (3-R) ribose structure, and can be separated from the 3-S isomer by chromatography.

The resulting product is cyclized by treatment with an acidic ion exchange resin, such as Dowex 50W-X12, to produce 2-deoxy-2,2-difluoro-D-erythro-pentanoic acid-γ-lactone 4. The hydroxy groups of the lactone are protected with tert-butyldimethylsilyl (TBDMS) protecting groups to obtain the protected lactone 3,5-bis-(tert-butyldimethylsilyloxy)-2-desoxy-2,2-difluoro-1-oxoribose 5, and the product is reduced to obtain 3,5-bis-(tert-butyldimethylsilyl)-2-desoxy-2,2-difluororibose 6.

The 1-position of the carbohydrate is activated by the introduction of a leaving group, e.g., methanesulfonyloxy (mesylate), formed by reacting compound 6 with methanesulfonyl chloride to obtain 3,5-bis-(tert-butyldimethylsilyloxy)-1-methanesulfonyloxy-2-desoxy-2,2-difluororibose 7. The base ring is coupled to the carbohydrate by reacting compound 7 with N,O-bis-(trimethylsilyl)-cytosine 8 in the presence of a reaction initiator, such as trifluoromethanesulfonyloxy trimethylsilane (trimethylsilyl triflate). Removal of the protecting groups and chromatographic purification affords gemcitabine free base.

U.S. Pat. No. 4,526,988 describes a similar process in which the cyclization is carried out by hydrolyzing an alkyl 3-dioxolanyl-2,2-difluoro-3-hydroxy-propionate with a mildly acidic ion exchange resin. See also, Hertel et al. in J. Org. Chem. 53, 2406 (1998).

U.S. Pat. No. 4,965,374 (“the '374 patent) describes a process for producing gemcitabine from an intermediate 3,5-dibenzoyl ribo protected lactone of the formula:

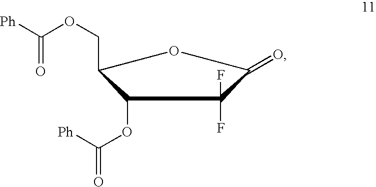

where the desired erythro isomer can be isolated in a crystalline form from a mixture of erythro and threo isomers. The process described in the '374 patent is generally outlined in Scheme 2.

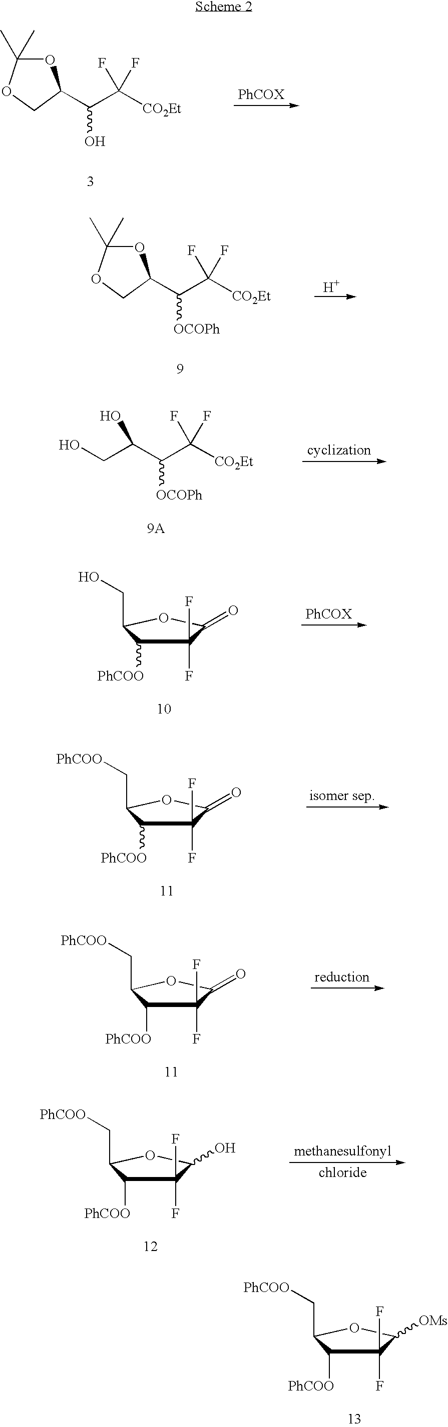

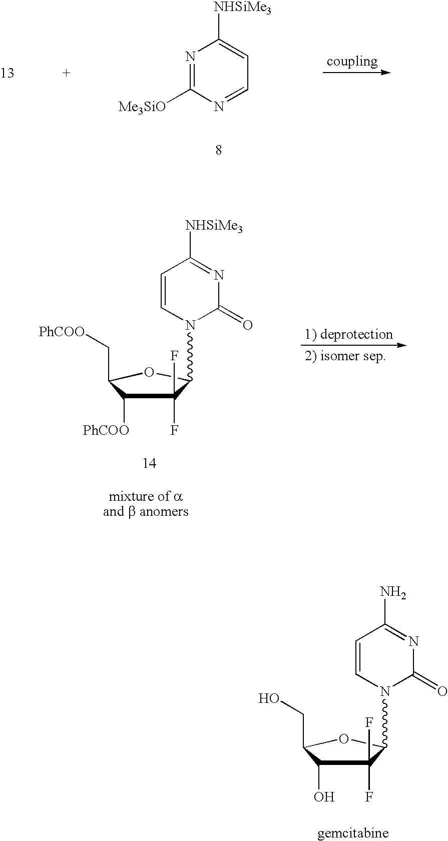

The 3-hydroxy group of compound 3 is esterified with a benzoyl protecting group by reaction with benzoyl chloride, benzoyl bromide, benzoyl cyanide, benzoyl azide, etc. (e.g., PhCOX, wherein X═Cl, Br, CN, or N₃), in presence of a tertiary amine or a catalyst such as 4-dimethylaminopyridine or 4-pyrrolidinopyridine, to obtain ethyl 2,2-difluoro-3-benzoyloxy-3-(2,2-dimethyldioxolan-4-yl)-propionate 9.

The isoalkylidene protecting group of 9 is selectively removed, e.g., by using a strong acid such as concentrated sulfuric acid in ethanol, to produce ethyl-2,2-difluoro-3-benzoyloxy-4,5-dihydroxypentanoate 9A. The product is cyclized to lactone 10 and converted to the dibenzoate ester to produce the lactone 2-deoxy-2,2-difluoropentofuranos-1-ulose-3,5-dibenzoate 11 as a mixture of erythro and threo isomers. The '374 patent describes isolating at least a portion of the erythro isomer from the mixture by selective precipitation. See also, Chou et al., Synthesis, 565-570, (1992).

Compound 11 is then reduced to obtain a mixture of α and β anomers of 2-desoxy-2,2-difluorpentofuranose-dibenzoate 12, which is activated with methane sulfonylchloride to obtain an anomeric mixture of mesylates, 2-deoxy-2,2-difluoro-D-ribofuranosyl-3,5-di-O-benzoyl-1-O-methanesulfonate 13, and coupled with N,O-bis(trimethylsilyl)-cytosine 8 to obtain silyl-protected nucleoside 14 as the dibenzoate ester as a mixture of the α- and β-anomers (about a 1:1 α/β anomer ratio). Removal of the esters and silyl protecting group provides a mixture of the β-anomer (gemcitabine) and the α-anomer (about a 1:1 α/β anomer ratio). The '374 patent describes selectively isolating the β-anomer (gemcitabine) by forming a salt of the anomeric mixture, e.g., the hydrochloride or hydrobromide salt, and selectively precipitating to obtain 2′-deoxy-2′,2′-difluorocytidine as the salt in 1:4 α/β ratio. The '374 patent also describes selectively precipitating the β-anomer in free base form in a slightly basic aqueous solution. One such process involves dissolving the 1:1 α/β anomeric mixture in hot acidic water (pH adjusted to 2.5-5.0) and, once the mixture is substantially dissolved, increasing the pH to 7.0-9.0 and allowing the solution to cool, to produce crystals, which are isolated by filtration.

Processes for separating anomeric mixtures of alkylsulfonate intermediates 13 also have been described. U.S. Pat. Nos. 5,256,797 and 4,526,988 describe processes for separating anomers of 2-deoxy-2,2-difluoro-D-ribofuranosyl-1-alkylsulfonates, and U.S. Pat. No. 5,256,798 describes a process for obtaining α-anomer-enriched ribofuranosyl sulfonates.

Other intermediates that may be useful for preparing gemcitabine have been disclosed. For instance, U.S. Pat. No. 5,480,992 describes anomeric mixtures of 2,2-difluororibosyl azide and corresponding amine intermediates that can be prepared, e.g., by reacting a 2-deoxy-2,2-difluoro-D-ribofuranosyl-3,5-di-O-benzoyl-1-O-β-methanesulfonate with an azide nucleophile, such as lithium azide, to obtain the azide. Reduction of the azide produces the corresponding amine, which can be synthetically converted into a nucleoside. See also U.S. Pat. Nos. 5,541,345 and 5,594,155.

Other known intermediates include, e.g., 1-alkylsulfonyl-2,2-difluoro-3-carbamoyl ribose and related nucleoside intermediates (U.S. Pat. No. 5,521,294), tritylated intermediates (U.S. Pat. No. 5,559,222), 2-deoxy 2,2-difluoro-β-D-ribo-pentopyranose (U.S. Pat. No. 5,602,262), 2-substituted-3,3-difluorofuran intermediates (U.S. Pat. No. 5,633,367), and α,α-difluoro-β-hydroxy thiol esters (U.S. Pat. Nos. 5,756,775 and 5,912,366).

There are inherent problems associated with the production of gemcitabine, particularly for processes that require the production and separation of isomers, which tend to produce poor yields on a commercial scale. Accordingly, there is a need for improved methods of preparing gemcitabine and intermediates thereof, which facilitate the production of gemcitabine, particularly on a commercial scale. The present invention provides such methods and intermediates, as will be apparent from the description of the invention provided herein.

### BRIEF SUMMARY OF THE INVENTION

The present invention provides compounds, which are useful intermediates for the production of gemcitabine, processes for producing such intermediates, and processes for producing gemcitabine therefrom. Exemplary intermediates of the present invention include 3-substituted, alkyl 2,2-difluoro-3-hydroxy-3-(2,2-dialkyldioxolan-4-yl)-propionate derivatives of the formula 15:

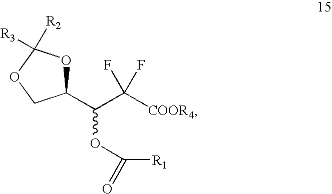

wherein R₁ is unsubstituted or substituted C₁-C₅ saturated or unsaturated alky, substituted phenyl, or C₁-C₅ saturated or unsaturated aralkyl; R₂ and R₃ are independently C₁-C₃ alkyl; and R₄ is C₁-C₄ alkyl. Exemplary intermediates of the present invention also include 3,5-disubstituted-2-deoxy-2,2-difluoro-1-oxo-D-ribose derivatives of the formula 16 and 16A:

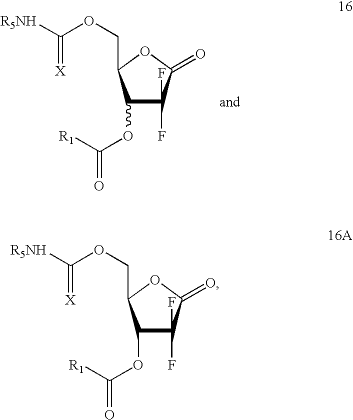

wherein R₁ is unsubstituted or substituted C₁-C₅ saturated or unsaturated alkyl, phenyl or substituted phenyl, or C₁-C₅ saturated or unsaturated aralkyl; X is O or S; and R₅ is unsubstituted or substituted phenyl, unsubstituted or substituted phenylsulfonyl, or C₁-C₅ alkylsulfonyl.

In accordance with the present invention, gemcitabine can be prepared from compounds of the formula 16A, which are readily obtainable from compounds of the formula 16. The present invention also provides a process for producing compounds of the formula 16 from compounds of the formula 15.

The diastereomeric mixtures of D-erythro-3,5-disubstituted-2-deoxy-2,2-difluoro-1-oxo-D-ribose derivatives of the formula 16, which are valuable as precursors in the synthesis of gemcitabine, are preferably prepared by hydrolyzing a mixture of erythro and threo isomers of alkyl 2,2-difluoro-3-hydroxy-3-(2,2-dialkyldioxolan-4-yl)-propionate of formula 15 using an acid as a hydrolytic reagent, followed by removal of water (preferably by azeotropic distillation) and reacting the resulting reaction mixture with a compound of the general formula R₅NCX (17), wherein X and R₅ are as defined herein. Preferably, compound 17 is an isocyanate or isothiocyanate.

The present invention further provides a process for selectively isolating the D-erythro-3,5-disubstituted-2-deoxy-2,2-difluoro-1-oxo-D-ribose of the formulae 16A:

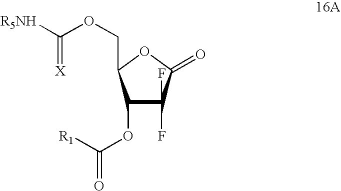

having purity of at least 95% from the diastereomeric mixtures of (D-erythro and D-threo)-3,5-disubstituted-2-deoxy-2,2-difluoro-1-oxo-D-ribose of the formula 16. An exemplary process includes mixing a diastereomeric mixture of (D-erythro and D-threo)-3,5-disubstituted-2-deoxy-2,2-difluoro-1-oxo-D-ribose with a non-polar solvent (e.g., to dissolve the diastereomeric mixture); cooling the mixture to promote crystallization; and collecting the crystals, e.g., by filtration, and optionally washing and/or drying the crystals.

### DETAILED DESCRIPTION OF THE PREFERRED EMBODIMENTS

The present invention provides compounds, which are useful intermediates for the production of gemcitabine, processes for producing such intermediates, and processes for producing gemcitabine therewith. Exemplary compounds of the present invention include 3-substituted, alkyl 2,2-difluoro-3-hydroxy-3-(2,2-dialkyldioxolan-4-yl)-propionate derivatives of the formula 15:

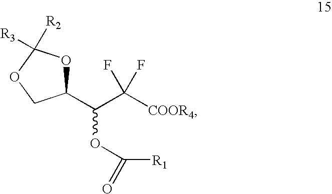

wherein R₁ is unsubstituted or substituted C₁-C₅ saturated or unsaturated alkyl, substituted phenyl, or C₁-C₅ saturated or unsaturated aralkyl; R₂ and R₃ are independently C₁-C₃ alkyl; and R₄ is C₁-C₄ alkyl. Exemplary compounds of the present invention also include 3,5-disubstituted-2-deoxy-2,2-difluoro-1-oxo-D-ribose derivatives of the formula 16 and 16A:

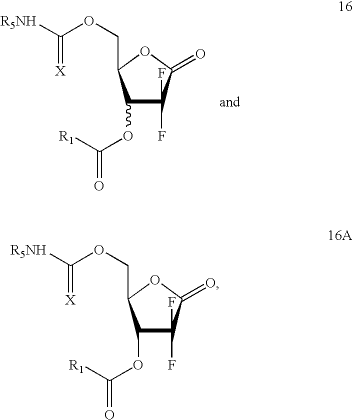

wherein R₁ is unsubstituted or substituted C₁-C₅ saturated or unsaturated alkyl, substituted phenyl, or C₁-C₅ saturated or unsaturated aralkyl; X is O or S; and R₅ is unsubstituted or substituted phenyl, unsubstituted or substituted phenylsulfonyl, or C₁-C₅ alkylsulfonyl.

In accordance with the present invention, R₁ includes C₁-C₅ saturated alkyl and C₁-C₅ unsaturated alkyl substituents, which may be unsubstituted or substituted phenyl (e.g., 4-chlorophenyl, which, when combined with the carbonyl, forms a 4-chlorobenzoyl), or C₁-C₅ saturated or unsaturated aralkyl (e.g., trans-2-phenylethenyl, which, when combined with the carbonyl, forms a cinnamoyl); R₂ and R₃ are the same or different and each can include, e.g., C₁-C₃ alkyl (e.g., methyl); and R₄ includes C₁-C₄ alkyl (e.g., ethyl); R₅ includes unsubstituted or substituted phenyl (e.g., phenyl, 4-methylphenyl, 4-chlorophenyl), unsubstituted or substituted phenylsulfonyl, or C₁-C₅ alkylsulfonyl; and X includes O or S.

In accordance with the present invention, gemcitabine can be prepared from compounds of the formula 16A, which are readily obtainable from compounds of formula 16. The present invention also provides methods of producing compounds of the formula 16 from compounds of the formula 15.

In a preferred embodiment, the present invention provides a method of converting a compound of the formula 16A into gemcitabine, as demonstrated in Scheme 3 below. An exemplary process of the present invention includes:

reducing a compound of the formula 16A:

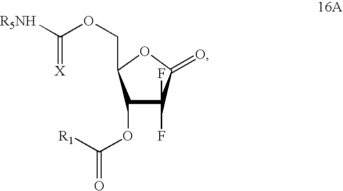

to produce a lactol of the formula 19:

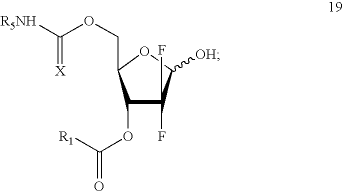

activating the hydroxyl group, e.g., by conversion into a sulfonate, e.g., a mesylate;

reacting the activated hydroxyl (e.g., the mesylate) with a suitably protected cytosine to produce a protected nucleoside;

optionally separating the β-anomer;

deprotecting the protected nucleoside; and,

optionally separating the β-anomer,

wherein R₁ is unsubstituted or substituted C₁-C₅ saturated or unsaturated alkyl, unsubstituted or substituted phenyl (e.g., 4-chlorophenyl), or C₁-C₅ saturated or unsaturated aralkyl (e.g., a trans-2-phenylethenyl, which, together with the carbonyl forms a cinnamoyl); R₅ is unsubstituted or substituted phenyl, unsubstituted or substituted phenylsulfonyl, or C₁-C₅ alkylsulfonyl; and X is O or S.

In another embodiment, the present invention provides processes for preparing the novel diastereomeric mixtures of (D-erythro and D-threo)-3,5-disubstituted-2-deoxy-2,2-difluoro-1-oxo-D-ribose derivatives of the formula 16, which are valuable as precursors in the synthesis of gemcitabine. In accordance with the present invention, compounds of formula 16 preferably are prepared by a process that includes hydrolyzing a mixture of erythro and threo isomers of a 3-substituted, alkyl 2,2-difluoro-3-hydroxy-3-(2,2-dialkyldioxolan-4-yl)-propionate in the presence of an acid, to produce a 3-substituted-2-deoxy-2,2-difluoro-1-oxo-D-ribose, and reacting the product with a compound of the formula R₅NCX (17), wherein X and R₅ are as defined herein. Optionally, water is removed from the reaction mixture produced in the hydrolysis step, preferably by azeotropic distillation. Preferably, compound 17 is an isocyanate or an isothiocyanate.

A preferred process of the present invention includes:

hydrolyzing a mixture of erythro and threo isomers of the 3-substituted, alkyl 2,2-difluoro-3-hydroxy-3-(2,2-dialkyldioxolan-4-yl)-propionate in the presence of a water-miscible solvent, water and an acid;

heating the mixture until the hydrolysis reaction is substantially complete;

optionally reducing the solution volume by distillation;

adding a water-immiscible solvent and removing at least a portion of the water (preferably removing at least a substantial portion of the water), e.g., by azeotropic distillation;

further distilling off the solvent mixture, to obtain a 3-substituted-2-deoxy-2,2-difluoro-1-oxo-D-ribose as a solid;

optionally treating the 3-substituted-2-deoxy-2,2-difluoro-1-oxo-D-ribose with activated carbon in an organic solvent and removing the activated carbon;

reacting the 3-substituted-2-deoxy-2,2-difluoro-1-oxo-D-ribose with an isocyanate or isothiocyanate, optionally in presence of a base, and mixing until the reaction is substantially complete; and

obtaining the resulting 3,5-disubstituted-2-deoxy-2,2-difluoro-1-oxo-D-ribose product by precipitation.

Exemplary water-miscible solvents include acetonitrile, tetrahydrofuran (THE), 2-methyltetrahydrofuran, acetone, N,N-dimethylformamide (DMF), N,N-dimethylacetamide (DMA), and mixtures thereof. A preferred water-miscible solvent is acetonitrile.

Exemplary acids include methanesulfonic acid, sulfuric acid, trifluoroacetic acid, and the like, and combinations thereof. A preferred acid is trifluoroacetic acid.

Exemplary mixtures of a water-miscible solvent, water and an acid preferably include mixtures of acetonitrile, water and trifluoroacetic acid. Exemplary acetonitrile:water:trifluoroacetic acid ratios include 54/1.25/0.3 v/v/v or 160/3.75/0.9 v/v/v (acetonitrile:water:trifluoroacetic acid).

Exemplary water-immiscible solvents include toluene, o-xylene, m-xylene, p-xylene, ethylbenzene, diethylbenzene, and the like, and mixtures thereof. A preferred water-immiscible solvent is toluene.

In accordance with the present invention, the isocyanates or isothiocyanates used in the reaction preferably are selected from, e.g., 2-chloroethyl isothiocyanate, 5-chloro-2-methylphenyl isothiocyanate, 2-chloro-4-nitrophenyl isothiocyanate, 2-chlorophenyl isothiocyanate, 3-chlorophenyl isothiocyanate, 4-chlorophenyl isothiocyanate, 3-acetylphenyl isothiocyanate, 4-acetylphenyl isothiocyanate, 2-(chloromethyl)phenyl isocyanate, 2-chloro-5-methyl-phenyl isocyanate, 2-chloro-6-methylphenyl isocyanate, 3-chloro-2-methylphenyl isocyanate, 3-chloro-4-methylphenyl isocyanate, 4-(chloromethyl)-phenyl isocyanate, 4-chloro-2-methylphenyl isocyanate, 5-chloro-2-methylphenyl isocyanate, 2-chloro-4-nitrophenyl isocyanate, 2-chloro-5-nitrophenyl isocyanate, 4-chloro-2-nitrophenyl isocyanate, 4-chloro-3-nitrophenyl isocyanate, 2-chloro-2-nitrophenyl isocyanate, 2-chlorophenyl isocyanate, 3-chlorophenyl isocyanate, 4-chlorophenyl isocyanate, 3-acetylphenyl isocyanate, phenyl isocyanate, N-benzenesulfonyl isocyanate, p-toluenesulfonyl isocyanate, and o-toluenesulfonyl isocyanate.

Preferably, the isocyanate or isothiocyanate includes p-toluenesulfonyl isocyanate, phenylsulfonyl isocyanate, or 4-chlorophenyl isothiocyanate.

The base that may be used in the reaction preferably includes triethyl amine, one or more lutidines, morpholine, diisopropylethylamine, pyridine, 2-(dimethylamino)-pyridine, 4-(dimethylamino)-pyridine, and the like, and combinations thereof. In a preferred embodiment, the base is 4-(dimethylamino)-pyridine.

The present invention further provides a process for selectively isolating the D-erythro-3,5-disubstituted-2-deoxy-2,2-difluoro-pentofuranose-1-oxo-D-ribose of formula 16A:

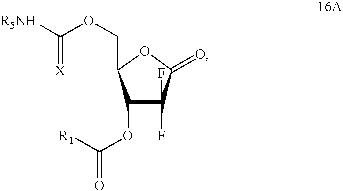

in purity of at least 95%, from a diastereomeric mixture of (D-erythro and D-threo)-3,5-disubstituted-2-deoxy-2,2-difluoro-1-oxo-D-ribose of the formula 16, the process comprising:

mixing the diastereomeric mixture with a non-polar solvent to dissolve at least a portion of the diastereomeric mixture;

cooling the mixture sufficiently to allow crystallization and isolating at least a portion of the crystals, e.g., by filtration, and, optionally, washing and/or drying the crystals.

Exemplary non-polar solvents, which can be used for precipitating and/or for washing include toluene, o-xylene, m-xylene, p-xylene, ethylbenzene, diethylbenzene, n-pentane, n-hexane, n-heptane, n-octane, isooctane, cyclohexane, petrol ether, and the like, and mixtures thereof. A preferred solvent used for precipitating is toluene, and preferred solvents for washing include toluene and hexane or a mixture thereof. An exemplary process of the present invention is depicted in Scheme 3.

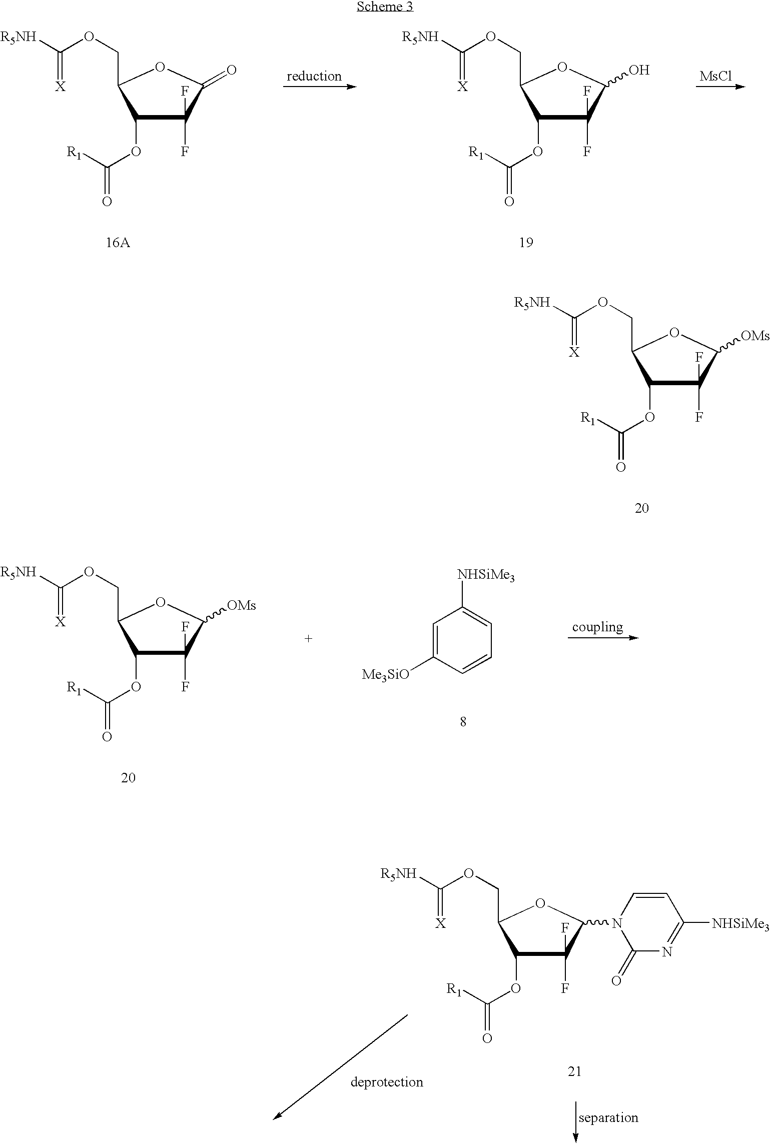

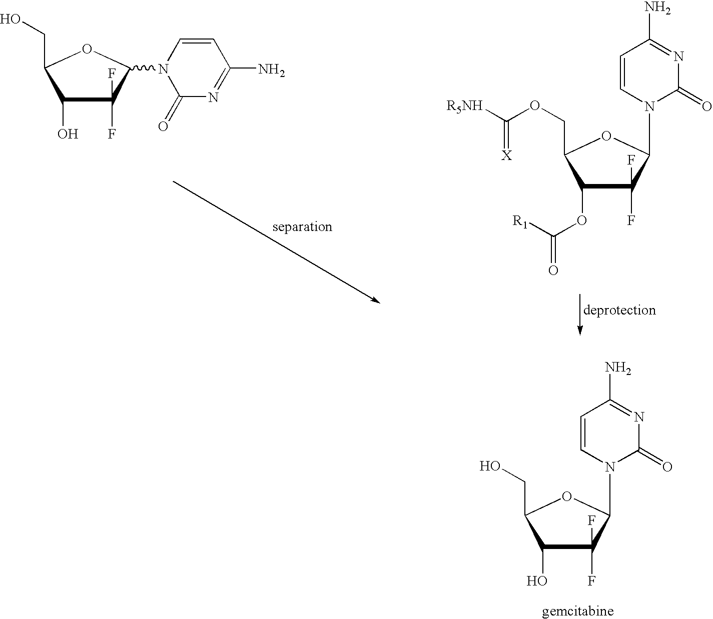

In accordance with Scheme 3, gemcitabine further can be obtained from the D-erythro-3,5-disubstituted-2-deoxy-2,2-difluoro-1-oxo-D-ribose of the formulae 16A by carrying out the following steps:

reducing the compound 16A with a suitable reducing agent in an organic solvent to obtain the lactol intermediate of the formula 19;

reacting the lactol intermediate of the formula 19 with methanesulfonyl chloride (MsCl) in the presence of a base to obtain the sulfonate intermediate of the formula 20;

coupling the compound 20 with bis(trimethylsilyl)-N-acetylcytosine 8, preferably at ambient temperatures using a catalyst in an organic solvent to obtain a mixture of the α and β anomers of the 3,5-diprotected-N-1-trimethylsilylacetyl-2′-deoxy-2′,2′-difluorocytidine 21;

optionally precipitating the β isomer of compound 21, thus separating the two isomers and allowing the β isomer to be isolated (e.g., by filtration); and

removing the protecting groups (e.g., by hydrolysis), to obtain gemcitabine; and

optionally separating the β anomer of gemcitabine.

In accordance with the present invention, the process using one of the starting materials D-erythro-3,5-disubstituted-2-deoxy-2,2-difluoro-1-oxo-D-ribose having the general formulae 16A (e.g., 3-cinnamoyloxy-5-(N-p-toluenesulfonyl)carbamoyloxy-2-deoxo-2,2-difluoro-1-oxo-D-ribose, 3-cinnamoyloxy-5-(N-benzenesulfonyl)-carbamoyloxy-2-deoxo-2,2-difluoro-1-oxo-D-ribose, 3-cinnamoyloxy-5-(N-4-chlorobenzenesulfonyl)-carbamoyloxy-2-deoxo-2,2-difluoro-1-oxo-D-ribose,3-cinnamoyloxy-5-(N-4-chlorophenyl)-carbamoyloxy-2-deoxo-2,2-difluoro-1-oxo-D-ribose, and 3-(4-chlorobenzoyloxy)-5-(N-4-chlorophenyl)-carbamoyloxy-2-deoxo-2,2-difluoro-1-oxo-D-ribose), e.g., as depicted in Scheme 3, is more advantageous over conventional processes for obtaining gemcitabine, as the present invention provides a process that requires fewer synthetic steps, and in addition the erythro isomer 16A is obtained in high purity and yield.

The reduction of the lactone 16A, e.g., as depicted in Scheme 3, can be carried out using any suitable reducing agent such as, for example, lithium aluminium hydride, diisobutyl aluminium hydride, and sodium bis-(2-methoxyethoxy)-aluminium hydride, or the like, or a combination thereof. The reduction, e.g., as illustrated in Scheme 3, is preferably carried out using lithium aluminium hydride, particularly for commercial scale production, particularly in view of its low molecular weight and relatively high reduction capacity (4 available hydrogen atoms per molecule). The reduction also can be carried out using diisobutyl aluminium hydride (e.g., as taught in U.S. Pat. No. 4,808,614 and Chou et al., Synthesis, 565-570 (1992), although diisobutyl aluminium hydride is less preferred in view of its molecular weight and the fact that it has only 1 hydrogen atom available for reduction.

The coupling reaction, e.g., as depicted in Scheme 3, can be carried out in any suitable solvent, which can include, for example, acetonitrile, dichloromethane, chloroform, 1,2-dichloroethane, toluene, one or more xylenes, and the like, and mixtures thereof. In one embodiment, the coupling reaction is carried out in 1,2-dichloroethane. Optionally, the coupling reaction can be facilitated by using a suitable catalytic reagent such as, for example, trimethylsilyl triflate (Me₃SiOTf).

Removal of the protecting groups, e.g., as depicted in Scheme 3, can be carried out by using any suitable conditions, which can include, for example, basic hydrolysis, e.g., ammonia in methanol.

### EXAMPLE 1

This example illustrates the preparation of 3-cinnamoyloxy-5-(N-p-toluenesulfonyl)-carbamoyloxy-2-deoxo-2,2-difluoro-1-oxo-D-ribose.

A mixture of ethyl (D-erythro and D-threo)-3-(cinnamoyloxy)-2,2-difluoro-3-(2,2-dimethyldioxolan-4-yl)-propionate [having a purity of 96% (by HPLC), a ratio of D-erythro-isomer to D-threo-isomer of 4.3 to 1; 15.6 g, 0.039 mol], acetonitrile (160 ml), CF₃COOH (0.9 ml) and water (3.75 ml) was heated under reflux for 5.5 hours. Then, the water/CF₃COOH/acetonitrile mixture (36 ml) was distilled off and toluene (36 ml) was added. A following portion (about 40 ml) was distilled and toluene (40 ml) was added. The procedure was repeated 4 times to obtain, while the internal temperature of the reaction mixture was about 99° C. Ethyl acetate (50 ml) and activated carbon (Darco G-60, 1.5 g) were added to the solution, and the mixture was heated under reflux for 0.5 hour. The activated carbon was collected by filtration to obtain a slightly yellow filtrate. The ethyl acetate was removed under reduced pressure, and the thus obtained residual solution was cooled to ambient temperature under nitrogen. Next, p-toluenesulfonyl isocyanate (96% purity, 8.5 g, 0.0429 mol, 1.1 equiv.) was added to the solution, and the reaction mixture was stirred at ambient temperature for 3 hours after which time a precipitate was obtained, and the mixture was kept at 5° C. overnight. The colorless precipitate was collected by filtration, washed with toluene and n-hexane and dried at 60° C. overnight to obtain 8.5 g of 3-cinnamoyloxy-5-(N-p-toluenesulfonyl)-carbamoyloxy-2-deoxy-2,2-difluoro-1-oxo-D-ribose, in 44.0% yield; mp.129-131° C. ¹H NMR (CDCl₃): δ=2.33 (s, 3 H, C₆H₄CH₃), 4.46 (2 AB-q, 2 H, CH₂), 4.72 (m, 1 H, 4-CH), 5.50 (m, 1 H, 3-CH), 6.45 (d, 1 H, ═CH), 7.34 (d, 2 Hₐᵣₒₘ, C₆H₄CH₃), 7.47 (m, 5 Hₐᵣₒₘ, C₆H₅), 7.79 (d, 1 H, ═CH), 7.91 (d, 2 Hₐᵣₒₘ, C₆H₄CH₃), 8.31 (s, 1 H, SO₂NHCO). ¹³C NMR (CDCl₃): δ=21.7 (C₆H₄CH₃), 63.1 (CH₂), 68.2 (C-3, J_{C-F}=30.0, 30.0 Hz), 77.3 (C-4), 111.2 (C-2, J_{C-F}=256, 256 Hz), 114.6 (═CH), 128.3, 128.5, 129.0, 129.8, 131.3, 133.5, 145.5 (Cₐᵣₒₘ), 148.7 (CH), 149.7 (OCONHSO₂), 162.1 (C-1, J_{C-F}=30, 30 Hz), 164.8 (OCOCH═CH). ¹⁹F NMR [δ=−118.6 (2 AB-q)], indicating that one fluorine containing product is mainly present. AOCI (negative)/MS: m/z=494.24 [M−H⁺].

### EXAMPLE 2

This example illustrates the preparation of 3-cinnamoyloxy-5-(N-benzenesulfonyl)-carbamoyloxy-2-deoxo-2,2-difluoro-1-oxo-D-ribose.

A mixture of ethyl (D-erythro and D-threo)-3-(cinnamoyloxy)-2,2-difluoro-3-(2,2-dimethyldioxolan-4-yl)-propionate [having a purity of 96% (by HPLC), a ratio of D-erythro-isomer to D-threo-isomer of 4.3 to 1; 5.2 g, 0.013 mol], acetonitrile (54 ml), CF₃COOH (0.3 ml) and water (1.25 ml) was heated under reflux for 5.5 hours. Then, the water/CF₃COOH/acetonitrile mixture (13 ml) was distilled off and toluene (13 ml) was added. A following portion (about 14 ml) was distilled and toluene (14 ml) was added. The procedure was repeated 4 times while the internal temperature of the reaction mixture was about 99° C. Ethyl acetate (20 ml) and activated carbon (Darco G-60, 0.3 g) were added to the solution, and the mixture was heated under reflux for 0.5 hour. The activated carbon was collected by filtration to obtain a slightly yellow filtrate. The ethyl acetate was removed under reduced pressure, and the residual solution was cooled to ambient temperature under nitrogen. Benzenesulfonyl isocyanate (95% purity, 2.6 g, 0.0135 mol, 1.04 equiv.) was then added to the solution, and the reaction mixture was stirred at ambient temperature overnight. A colorless precipitate was collected by filtration, washed with toluene and n-hexane and dried at 60° C. overnight to obtain 2.2 g of the pure 3-cinnamoyloxy-5-(N-benzenesulfonyl)-carbamoyloxy-2-deoxy-2,2-difluoro-1-oxo-D-ribose in 35.2% yield, [α]_{D}²⁵+51.1° (c 1, in acetonitrile); mp.145-146.5° C. ¹H NMR (CDCl₃): δ=4.51 (2 AB-q, 2 H, CH₂), 4.76 (q, 1 H, 4-CH), 5.55 (m, 1 H, 3-CH), 6.49 (d, 1 H, ═CH), 7.47 (m, 3 Hₐᵣₒₘ,), 7.60 (m, 4 Hₐᵣₒₘ,), 7.71 (t, 1 Hₐᵣₒₘ), 7.83 (d, 1 H, ═CH), 8.08 (d, 2 Hₐᵣₒₘ), 8.30 (s, 1 H, SO₂NHCO).

¹³C NMR (CDCl₃): δ=63.4 (CH₂), 68.3 (C-3, J_{C-F}=30.0, 30.0 Hz), 77.5 (C-4),111.4 C-2, J_{C-F}=256, 256 Hz), 114.8 (═CH), 128.4, 128.7, 129.2, 129.4, 131.5, 133.6, 134.4, 138.1 (Cₐᵣₒₘ), 148.9 (═CH), 149.7 (OCONHSO₂), 162.1 (C-1, J_{C-F}=30, 30 Hz), 165.0 (OCOCH═CH). ESI (negative)/MS: m/z=480.1 [M−H⁺].

### EXAMPLE 3

This example illustrates the preparation of 3-cinnamoyloxy-5-(N-4-chlorobenzenesulfonyl)-carbamoyloxy-2-deoxo-2,2-difluoro-1-oxo-D-ribose.

A mixture of ethyl (D-erythro and D-threo)-3-(cinnamoyloxy)-2,2-difluoro-3-(2,2-dimethyldioxolan-4-yl)-propionate [having a purity of 96% (by HPLC), a ratio of D-erythro-isomer to the D-threo-isomer of 4.3 to 1; 5.2 g, 0.013 mole], acetonitrile (54 ml), CF₃COOH (0.3 ml) and water (1.25 ml) was heated under reflux for 5.5 hours. Then, the water/CF₃COOH/acetonitrile mixture (13 ml) was distilled off and toluene (13 ml) was added. A following portion (about 14 ml) was distilled and toluene (14 ml) was added. The procedure was repeated 4 times, while the internal temperature of the reaction mixture was about 99° C. Ethyl acetate (20 ml) and activated carbon (Darco G-60, 0.3 g) were added to the residual solution, and the mixture was heated under reflux for 0.5 hour. The activated carbon was collected by filtration to obtain a slightly yellow filtrate. The ethyl acetate was removed under reduced pressure, and the residual solution was cooled to ambient temperature under nitrogen. 4-chlorobenzenesulfonyl isocyanate (97% purity, 3.2 g, 0.0143 mole, 1.1 equiv.) was then added to the solution, and the reaction mixture was stirred at ambient temperature overnight. The reaction mixture was then kept at −20° C. for 78 hours. A colorless precipitate was collected by filtration, washed with cold toluene and n-hexane and dried at 60° C. overnight to obtain 3.1 g of pure 3-cinnamoyloxy-5-(N-4-chlorobenzene-sulfonyl)-carbamoyloxy-2-deoxy-2,2-difluoro-1-oxo-D-ribose in 43.6% yield. The crude product was dissolved in toluene (3.5 ml) and the solution was kept at 5° C. overnight. The colorless crystals were collected by filtration to give a pure 3-cinnamoyloxy-5-(N-4-chlorobenzenesulfonyl)-carbamoyloxy-2-deoxy-2,2-difluoro-1-oxo-D-ribose; total yield: 2.0 g (29.9%); [α]_{D}²⁵+29.3° (c 1, acetonitrile); mp 145-147° C. ¹H NMR (CDCl₃): δ=4.49 (2 AB-q, 2 H, CH₂), 4.76(q, 1 H, 4-CH), 5.57 (m, 1 H, 3-CH), 6.43 (d, 1 H, ═CH), 7.41(m, 3 Hₐᵣₒₘ), 7.50 (m, 4 Hₐᵣₒₘ), 7.76 (d, 1 H, ═CH), 7.95 (d, 2 Hₐᵣₒₘ), 8.89 (s, 1 H, SO₂NHCO). ¹³C NMR (CDCl₃): δ=63.3 (CH₂), 68.2 (C-3, J_{C-F}=30.0, 30.0 Hz), 77.4 (C-4, J_{C-F}=7 Hz), 111.3 (C-2, J_{C-F}=256, 256 Hz), 114.6 (═CH), 128.6, 129.1, 129.5, 129.8, 131.4, 133.4, 136.3, 141.0 (Cₐᵣₒₘ), 148.7 (═CH), 149.9 (OCONHSO₂), 162.4 (C-1, J_{C-F}=30, 30 Hz), 165.1 (OCOCH═CH). The ¹⁹F NMR spectrum indicates that one fluorine containing product is mainly present. APCI (positive)/MS: m/z=516.14 [M+H]⁺.

### EXAMPLE 4

This example illustrates the reparation of 3-cinnamoyloxy-5-(N-4-chlorophenyl)-carbamoyloxy-2-deoxo-2,2-difluoro-1-oxo-D-ribose.

A mixture of ethyl (D-erythro and D-threo)-3-(cinnamoyloxy)-2,2-difluoro-3-(2,2-dimethyldioxolan-4-yl)-propionate [having a purity of 96% by HPLC, a ratio of D-erythro-isomer to D-threo-isomer of 4.3 to 1; 5.2 g, 0.013 mole], acetonitrile (54 ml), CF₃COOH (0.3 ml) and water (1.25 ml) was heated under reflux for 5.5 hours. Then, water/CF₃COOH/acetonitrile mixture (13 ml) was distilled off and toluene (13 ml) was added. A following portion (about 14 ml) was distilled and toluene (14 ml) was added. The procedure was repeated 4 times, while the internal temperature of the reaction mixture was about 99° C. Ethyl acetate (20 ml) and an activated carbon (Darco G-60, 0.3 g) were added to the residual solution, and the mixture was heated under reflux for 0.5 hour. The activated carbon was collected by filtration to obtain a slightly yellow filtrate. The ethyl acetate was removed from the filtrate under reduced pressure, and the residual solution was cooled to ambient temperature under nitrogen. 4-chlorophenyl isocyanate (98% purity, 2.48 g, 0.0143 mol, 1.1 equiv.) and 4-(dimethylamino)-pyridine (99% purity, 0.033 g, 0.0003 mole) were then added to the solution, and the reaction mixture was stirred at 80-90° C. for 6 hours, cooled to ambient temperature, and the colorless crystals of 1,3-di(4-chlorophenyl)urea were collected by filtration. Ethyl acetate (20 ml) and an activated carbon (Darco G-60, 0.3 g) were added to the filtrate and the mixture was heated under reflux for 0.5 hour. The activated carbon was collected by filtration to obtain a slightly yellow filtrate. The solvents were removed under reduced pressure from the filtrate to yield 5.64 g of crude (D-erythro and D-threo)-3-cinnamoyloxy-5-(N-4-chlorophenylcarbamoyloxy)-2-deoxo-2,2-difluoropentofuranos-1-ulose cake in 96% yield. Toluene (12 ml) was added to the cake and the mixture was heated to obtain a solution. The solution was kept at 5° C. overnight. A colorless precipitate was collected by filtration, washed with toluene and n-hexane and dried at 60° C. overnight to obtain 2.7 g of pure 3-cinnamoyloxy-5-(N-4-chlorophenyl)-carbamoyloxy-2-deoxy-2,2-difluoro-1-oxo-D-ribose in 46% yield; [α]_{D}²⁵+95.0° (c 1, in acetonitrile); mp. 119-121° C. ¹H NMR (CDCl₃): δ=4.59 (2 AB-q, 2 H, CH₂), 4.88 (q, 1 H, 4-CH), 5.67 (m, 1 H, 3-CH), 6.51 (d, 1 H, ═CH), 7.14 (s, 1 H, ArNHCO), 7.32 (m, 4 Hₐᵣₒₘ,), 7.51 (m, 5 Hₐᵣₒₘ), 7.84 (d, 1 H, ═CH). ¹³C NMR (CDCl₃): δ=62.3 (CH₂), 68.7 (C-3, J_{C-F}=30.0, 30.0 Hz), 78.4 (C-4, J_{C-F}=6 Hz), 111.6 (C-2, J_{C-F}=256, 256 Hz), 114.8 (═CH), 120.3, 128.6, 129.1, 129.2, 131.5, 133.5, 135.8 (Cₐᵣₒₘ), 148.7 (═CH), 152.3 (OCONHAr), 162.8 (C-1, J_{C-F}=30, 30 Hz), 165.0 (OCOCH═CH). The ¹⁹F NMR spectrum indicates that one fluorine containing product is mainly present. ESI (positive)/MS: m/z=451.44[M+H]⁺.

### EXAMPLE 5

This example illustrates the preparation of 3-(4-chlorobenzoyloxy)-5-(N-4-chlorophenyl)-carbamoyloxy-2-deoxo-2,2-difluoro-1-oxo-D-ribose.

A mixture of ethyl (D-erythro and D-threo)-3-hydroxy-2,2-difluoro-3-(2,2-dimethyldioxolan-4-yl)-propionate [having a purity of 83% (by HPLC), a ratio of the D-erythro-isomer to the D-threo-isomer of 3.4 to 1; 6.25 g, 0.02 mol], 2,6-lutidine (4.65 ml, 0.04 mol) and 4-(dimethylamino)-pyridine (1.2 g, 0.01 mol) in ethyl acetate (30 ml) was warmed to 65-70° C. Then, a solution of 4-chlorobenzoyl chloride (3.05 ml, 0.024 mol) in ethyl acetate (25 ml) was added drop-wise for 4 hours at this temperature. The mixture was cooled to 5° C. and 2,6-lutidine hydrochloride was filtered off. The ethyl acetate was removed under reduced pressure from the filtrate to obtain 7.6 g of ethyl (D-erythro and D-threo)-3-(4-chlorobenzoyloxy)-2,2-difluoro-3-(2,2-dimethyldioxolan-4-yl)-propionate as an oil in 97% yield. Acetonitrile (82 ml), CF₃COOH (0.5 ml) and water (1.9 ml) were added to the oil and the mixture was heated under reflux for 5.5 hours. Then, the water/CF₃COOH/acetonitrile mixture (20 ml) was distillated and toluene (20 ml) was added. A following portion (about 20 ml) was distilled and toluene (20 ml) was added. The procedure was repeated 4 times to obtain a temperature of the reaction mixture of about 99° C. Ethyl acetate (20 ml) and activated carbon (Darco G-60, 0.4 g) were added to the residual solution, and the mixture was heated under reflux for 0.5 hour. The activated carbon was collected by filtration to obtain a slightly yellow filtrate. The ethyl acetate was removed from the filtrate under reduced pressure, and the residual solution was cooled to ambient temperature under nitrogen. 4-chlorophenyl isocyanate (98% purity, 3.45 g, 0.022 mol, 1.1 eq.) and 4-(dimethylamino)-pyridine (99% purity, 0.050 g, 0.0004 mol) were then added to the solution, and the reaction mixture was stirred at 80-90° C. for 6 hours, cooled to ambient temperature, and the colorless crystals of 1,3-di(4-chlorophenyl)urea were collected by filtration. Ethyl acetate (20 ml) and activated carbon (Darco G-60, 0.4 g) were added to the filtrate and the mixture was heated under reflux for 0.5 hour. The activated carbon was collected by filtration to obtain a slightly yellow filtrate. The solvents were removed under reduced pressure and toluene (22 ml) was added to residual oil (11 g). The mixture was heated to obtain a solution. The solution was kept at 5° C. overnight. A colorless precipitate was collected by filtration, washed with toluene and n-hexane and dried at 60° C. overnight to yield 2.75 g of 3-cinnamoyloxy-5-(N-4-chlorophenyl)-carbamoyloxy-2-deoxy-2,2-difluoro-1-oxo-D-ribose in 30.4% yield; [α]_{D}²⁵+91.7° (c 1, in acetonitrile); mp. 136.5-138.0° C.

¹H NMR (CDCl₃): δ=4.58 (2 AB-q, 2 H, CH₂), 4.90 (q, 1 H, 4-CH), 5.70 (m, 1 H, 3-CH), 7.00 (s, 1 H, ArNHCO), 7.28 (m, 4 Hₐᵣₒₘ,), 7.45 (d, 2 Hₐᵣₒₘ), 7.98 (d, 2 Hₐᵣₒₘ).

¹³C NMR (CDCl₃): δ=62.2 (CH₂), 69.2 (C-3, J_{C-F}=30.0, 30.0 Hz), 78.3 (C-4, J_{C-F}=6 Hz), 111.4(C-2, J_{C-F}=256, 256 Hz), 120.2, 125.7, 129.1, 129.2, 131.5, 135.6, 141.3 (Cₐᵣₒₘ), 152.1 (OCONHAr), 162.5 (C-1, J_{C-F}=30, 30 Hz), 163.9 (OCOAr). The ¹⁹F NMR spectrum indicates that one fluorine containing product is mainly present APCI (positive)/MS: m/z=459.6 [M+H]⁺.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A process for preparing gemcitabine, the process comprising:
reducing a compound of the formula 16A: 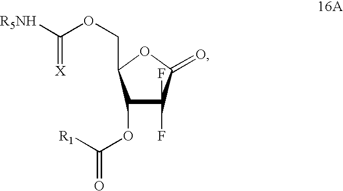
to produce a compound of the formula 19: 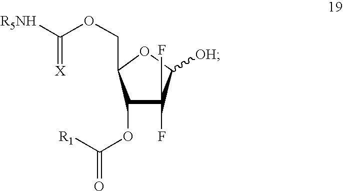
activating the hydroxyl group e.g., by conversion into a sulfonate (mesylate);
reacting the activated hydroxyl (mesylate) with a protected cytosine to produce a protected nucleoside;
optionally separating the β anomer;
deprotecting the protected nucleoside; and,
optionally separating the βanomer,
wherein R₁ is unsubstituted or substituted C₁-C₅ saturated or unsaturated alkyl, substituted phenyl, or C₁-C₅ saturated or unsaturated aralkyl; R₅ is unsubstituted or substituted phenyl, unsubstituted or substituted phenylsulfonyl, or C₁-C₅ alkylsulfonyl; and X is O or S.

2. A 3-substituted, alkyl 2,2-difluoro-3-hydroxy-3-(2,2-dialkyldioxolan-4-yl)-propionate of the formula 15: 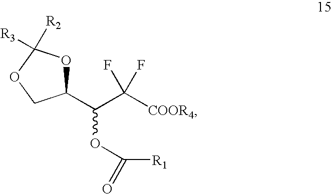
wherein R₁ is unsubstituted or substituted C₁-C₅ saturated or unsaturated alkyl, substituted phenyl, or C₁-C₅ saturated or unsaturated aralkyl; R₂ and R₃ are independently C₁-C₃ alkyl; and R₄ is C₁-C₄ alkyl.

3. A 3,5-disubstituted-2-deoxy-2,2-difluoro-1-oxo-D-ribose of the formula 16: 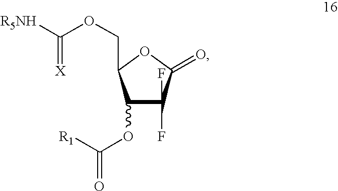
wherein R₁ is an unsubstituted or substituted C₁-C₅ saturated or unsaturated alkyl, substituted phenyl, or C₁-C₅ saturated or unsaturated aralkyl; X is O or S; and R₅ is unsubstituted or substituted phenyl, unsubstituted or substituted phenylsulfonyl, or C₁-C₅ alkylsulfonyl.

4. A process for preparing the compound of formula 16, the process comprising:
hydrolyzing a mixture of erythro and threo isomers of a 3-substituted, alkyl 2,2-difluoro-3-hydroxy-3-(2,2-dialkyldioxolan-4-yl)-propionate in the presence of an acid, to produce a 3-substituted-2-deoxy-2,2-difluoro-1-oxo-D-ribose; and
reacting the 3-substituted-2-deoxy-2,2-difluoro-1-oxo-D-ribose with a compound of the formula R₅NCX (17), optionally in presence of a base, wherein X is O or S, and R₅ is unsubstituted or substituted phenyl, unsubstituted or substituted phenylsulfonyl, or C₁-C₅ alkylsulfonyl.

5. The process of claim 4, wherein the 3-substituted, alkyl 2,2-difluoro-3-hydroxy-3-(2,2-dialkyldioxolan-4-yl)-propionate is a compound of the formula 15: 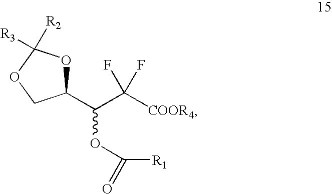
wherein R₁ is unsubstituted or substituted C₁-C₅ saturated or unsaturated alkyl, phenyl, substituted phenyl, or C₁-C₅ saturated or unsaturated aralkyl; R₂ and R₃ are independently C₁-C₃ alkyl; and R₄ is C₁-C₄ alkyl.

6. The process of claim 5, wherein the 3-substituted, alkyl 2,2-difluoro-3-hydroxy-3-(2,2-dialkyldioxolan-4-yl)-propionate of the formula 15 is ethyl (D-erythro and D-threo)-3-(cinnamoyloxy)-2,2-difluoro-3-(2,2-dimethyldioxolan-4-yl)-propionate or ethyl (D-erythro and D-threo)-3-(4-chlorobenzoyloxy)-2,2-difluoro-3-(2,2-dimethyldioxolan-4-yl)-propionate.

7. The process of claim 4, further comprising removing water from the reaction mixture containing the 3-substituted-2-deoxy-2,2-difluoro-1-oxo-D-ribose.

8. The process of claim 4, comprising:
hydrolyzing the mixture of erythro and threo isomers of the 3-substituted, alkyl 2,2-difluoro-3-hydroxy-3-(2,2-dialkyldioxolan-4-yl)-propionate in the presence of a water-miscible solvent, water and an acid;
heating the mixture until the hydrolysis reaction is substantially complete;
optionally reducing the solution volume by distillation;
adding a water-unmiscible solvent and removing the water;
further distilling off the solvent mixture to obtain the 3-substituted-2-deoxy-2,2-difluoro-1-oxo-D-ribose as a solid;
optionally treating the 3-substituted-2-deoxy-2,2-difluoro-1-oxo-D-ribose with activated carbon in an organic solvent and removing the activated carbon;
reacting the 3-substituted-2-deoxy-2,2-difluoro-1-oxo-D-ribose with an isocyanate or isothiocyanate until the reaction is substantially complete; and
precipitating the 3,5-disubstituted-2-deoxy-2,2-difluoro-1-oxo-D-ribose.

9. The process of claim 8, wherein the water-miscible solvent is acetonitrile, tetrahydrofuran (THF), 2-methyltetrahydrofuran, acetone, N,N-dimethyl formamide (DMF), N,N-dimethyl acetamide (DMA), or a mixture thereof.

10. The process of claim 9, wherein the water-miscible solvent is acetonitrile.

11. The process of claim 8, wherein the acid is methanesulfonic acid, sulfuric acid, trifluoroacetic acid, or a combination thereof.

12. The process of claim 11, wherein the acid is trifluoroacetic acid.

13. The process of claim 10, wherein the 3-substituted, alkyl 2,2-difluoro-3-hydroxy-3-(2,2-dialkyldioxolan-4-yl)-propionate is hydrolyzed in a mixture of acetonitrile, water and trifluoroacetic acid.

14. The process of claim 7, wherein water is removed by azeotropic distillation.

15. The process of claim 8, wherein the water-immiscible solvent is toluene, o-xylene, m-xylene, p-xylene, ethylbenzene, diethylbenzene, or a mixture thereof.

16. The process of claim 15, wherein the water-immiscible solvent is toluene.

17. The process of claim 8, wherein the isocyanate or isothiocyanate is 2-chloroethyl isothiocyanate, 5-chloro-2-methylphenyl isothiocyanate, 2-chloro-4-nitrophenyl isothiocyanate, 2-chlorophenyl isothiocyanate, 3-chlorophenyl isothiocyanate, 4-chlorophenyl isothiocyanate, 3-acetylphenyl isothiocyanate, 4-acetylphenyl isothiocyanate, 2-(chloromethyl)phenyl isocyanate, 2-chloro-5-methyl-phenyl isocyanate, 2-chloro-6-methylphenyl isocyanate, 3-chloro-2-methylphenyl isocyanate, 3-chloro-4-methylphenyl isocyanate, 4-(chloromethyl)-phenyl isocyanate, 4-chloro-2-methylphenyl isocyanate, 5-chloro-2-methylphenyl isocyanate, 2-chloro-4-nitrophenyl isocyanate, 2-chloro-5-nitrophenyl isocyanate, 4-chloro-2-nitrophenyl isocyanate, 4-chloro-3-nitrophenyl isocyanate, 2-chloro-2-nitrophenyl isocyanate, 2-chlorophenyl isocyanate, 3-chlorophenyl isocyanate, 4-chlorophenyl isocyanate, 3-acetylphenyl isocyanate, phenyl isocyanate, N-benzenesulfonyl isocyanate, p-toluenesulfonyl isocyanate, or o-toluenesulfonyl isocyanate.

18. The process of claim 17, wherein the isocyanate or isothiocyanate is p-toluenesulfonyl isocyanate, phenylsulfonyl isocyanate, or 4-chlorophenyl isothiocyanate.

19. The process of claim 4, wherein the base is triethyl amine, a lutidine, morpholine, diisopropylethylamine, pyridine, 2-(dimethylamino)-pyridine, 4-(dimethylamino)-pyridine, or a combination thereof.

20. The process of claim 19, wherein the base is 4-(dimethylamino)-pyridine.

21. A process for preparing D-erythro-3,5-disubstituted-2-deoxy-2,2-difluoro-1-oxo-D-ribose of the formula 16A: 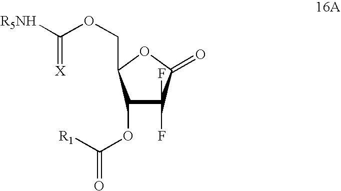
having purity of at least 95%, the process comprising:
dissolving in a non-polar solvent a diastereomeric mixture of (D-erythro and D-threo)-3,5-disubstituted-2-deoxy-2,2-difluoro-1-oxo-D-ribose of the formula 16: 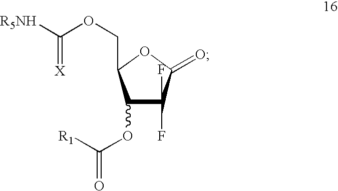
cooling the mixture sufficiently to produce crystals of a compound of the formula 16A;
collecting a least a portion of the crystals;
optionally washing the crystals; and
optionally drying the crystals.

22. The process of claim 21, wherein the non-polar solvent is toluene, o-xylene, m-xylene, p-xylene, ethylbenzene, diethylbenzene, n-pentane, n-hexane, n-heptane, n-octane, isooctane, cyclohexane, petrol ether, or a mixture thereof.

23. The process of claim 22, wherein the non-polar solvent comprises toluene.

24. The process of claim 22, wherein the non-polar solvent comprises a mixture of toluene and n-hexane.

25. A D-erythro-3,5-disubstituted-2-deoxy-2,2-difluoro-1-oxo-D-ribose, which is 3-cinnamoyloxy-5-(N-p-toluenesulfonyl)carbamoyloxy-2-deoxo-2,2-difluoro-1-oxo-D-ribose, 3-cinnamoyloxy-5-(N-benzene-sulfonyl)-carbamoyloxy-2-deoxo-2,2-difluoro-1-oxo-D-ribose, 3-cinnamoyloxy-5-(N-4-chlorobenzenesulfonyl)-carbamoyloxy-2-deoxo-2,2-difluoro-1-oxo-D-ribose, 3-cinnamoyloxy-5-(N-4-chlorophenyl)-carbamoyloxy-2-deoxo-2,2-difluoro-1-oxo-D-ribose, or 3-(4-chlorobenzoyloxy)-5-(N-4-chlorophenyl)-carbamoyloxy-2-deoxo-2,2-difluoro-1-oxo-D-ribose.
